(19) United States
(12) Patent Application Publication
(10) Pub. No.: US 2026/0294507 A1
(43) Pub. Date: Oct. 1, 2026

(54) POWER LIMITING OF RF DELIVERY DURING RF PUNCTURE

(71) Applicant: Boston Scientific Scimed, Inc., Maple Grove, MN (US)

(72) Inventors: Steven Kinio, Pickering (CA); German Ruvalcaba Alvarez, Toronto (CA); Christian Balkovec, Cambridge (CA); Cynthia Leung, Toronto (CA); Gareth Davies, Toronto (CA); Deep Patel, Waterdown (CA)

(21) Appl. No.: 19/465,730

(22) Filed: Jan. 30, 2026

Related U.S. Application Data
(60) Provisional application No. 63/752,566, filed on Jan. 31, 2025.

(51) Int. Cl. A61B 18/12; A61B 18/00; A61B 18/14; A61B 18/16

(52) U.S. Cl. CPC A61B 18/1206; A61B 18/1492; A61B 18/16; A61B 2018/00357; A61B 2018/00577; A61B 2018/00702; A61B 2018/00767; A61B 2018/00791; A61B 2018/167

(57) ABSTRACT

A radiofrequency (RF) generator for use in a medical system is disclosed. The RF generator includes and RF energy source and a controller. The RF energy source includes an active connector and a return connector. The active connector is coupled to an active electrode of an RF puncture device and the return connector is coupled to a return electrode. The active electrode and return electrode are coupled to a body. The controller is coupled to the RF energy source. The controller applies an RF puncture signal based on a feedforward control setting, asserts an electrical power control of the puncture signal based on thermal energy to the tissue applied with the RF puncture signal, and monitors the puncture signal for desiccation and fulguration of the tissue based on thermal energy applied to the tissue with the RF puncture signal.

### CROSS REFERENCE TO RELATED APPLICATIONS

This application claims priority to U.S. Provisional Patent Application No. 63/752,566 entitled “POWER LIMITING OF RF DELIVERY DURING RF PUNCTURE,” filed Jan. 31, 2025, which is hereby incorporated by reference in its entirety.

### TECHNICAL FIELD

The present disclosure relates to medical devices and systems for use in percutaneous or interventional procedures including electrosurgery such as electrophysiology procedures. More specifically, this disclosure relates to electrosurgical units, such as radiofrequency (RF) generators, electrosurgical systems, and methods, that provide for impedance measurements.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools to create the passage. The transseptal puncture permits a direct route to the left atrium via the atrial septum and systematic venous system. Increasing larger and complex medical devices can be passed into the left atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of crossing devices having electrodes energized with a suitable power source such as an electrically coupled electrosurgical generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to puncture tissue with plasma formed on the energized electrode. Tissue that contacts the plasma experiences a rapid vaporization of cellular fluid to produce a puncturing effect. Electrical energy from the electrosurgical generator can be applied to the electrodes either as a train of high frequency pulses or as a continuous signal typically in the radiofrequency (RF) range to perform the puncturing techniques.

### SUMMARY

In Example 1, a radiofrequency (RF) generator for use in a medical system, the RF generator comprising: an RF energy source having an active connector and a return connector, the active connector configured to be coupled to an active electrode of an RF puncture device and the return connector configured to be coupled to a return electrode, the active electrode and return electrode configured to couple to a body; and a controller coupled to the RF energy source, the controller configured to: apply an RF puncture signal based on a feedforward control setting, assert an electrical power control of the puncture signal based on thermal energy to the tissue applied with the RF puncture signal, and monitor the puncture signal for desiccation and fulguration of the tissue based on thermal energy applied to the tissue with the RF puncture signal.

In Example 2, the RF generator of Example 1, wherein the feedforward control setting is based on a nominal feedforward control setting stored in a non-transitory memory device.

In Example 3, the RF generator of Example 2, wherein the feedforward control setting is an adjusted nominal feedforward control setting.

In Example 4, the RF generator of any of Examples 1-3, comprising an RF output circuit coupled to the controller and wherein the electrical power control is asserted via disabling an RF output circuit.

In Example 5, the RF generator of Example 4, wherein the RF output circuit includes a voltage converter, and the voltage converter is disabled based on electrical current drawn by the voltage converter.

In Example 6, the RF generator of Example 5, wherein the RF output circuit includes a comparator circuit coupled to the voltage converter, and the electrical current and a reference current value are provided to a comparator circuit.

In Example 7, the RF generator of Example 5, comprising a measurement circuit coupled to the RF output circuit and to the controller, wherein the measurement circuit is applied to determine the electrical current and provide a digitized signal to the controller.

In Example 8, the RF generator of any of Examples 4-7, wherein the electrical power is one of an apparent electrical power or a real electrical power.

In Example 9, the RF generator of Example 4, wherein the electrical power control is asserted via disabling the RF output circuit based on a thermal signal.

In Example 10, the RF generator of Example 9, comprising a thermal sensor connector and wherein the RF puncture device includes a thermal sensor proximate the active electrode.

In Example 11, the RF generator of Example 10, comprising a measurement circuit coupled to the thermal sensor connector and to the controller.

In Example 12, the RF generator of Example 1, wherein the controller is configured to assert the electrical power based on one of a power of the RF puncture signal or a voltage of the RF puncture signal.

In Example 13, the RF generator of any of Examples 1-12, wherein the controller is configured to monitor the puncture signal for desiccation based on time of insufficient power of the RF puncture signal.

In Example 14, the RF generator of any of Examples 1-13, wherein the controller is configured to monitor the puncture signal for fulguration based on time of sufficient power of the RF puncture signal.

In Example 15, the radiofrequency generator of any of Examples 1-14, wherein the RF puncture device is an electrosurgical transseptal guidewire.

In Example 16, a radiofrequency (RF) generator for use in a tissue puncture system for puncturing a tissue in a body, the RF generator comprising: an RF energy source having an active connector and a return connector, the active connector configured to be coupled to an active electrode of an RF puncture device and the return connector configured to be coupled to a return electrode, the active electrode and return electrode configured to couple to the body; and a controller coupled to the RF energy source, the controller configured to: apply an RF puncture signal based on a feedforward control setting, assert an electrical power control of the puncture signal based on thermal energy to the tissue applied with the RF puncture signal, and monitor the puncture signal for desiccation and fulguration of the tissue based on thermal energy applied to the tissue with the RF puncture signal.

In Example 17, the RF generator of Example 16, wherein the feedforward control setting is based on a nominal feedforward control setting stored in a non-transitory memory device.

In Example 18, the RF generator of Example 17, wherein the feedforward control setting is an adjusted nominal feedforward control setting.

In Example 19, the RF generator of Example 16, comprising an RF output circuit coupled to the controller and wherein the electrical power control is asserted via disabling an RF output circuit.

In Example 20, the RF generator of Example 19, wherein the RF output circuit includes a voltage converter, and the voltage converter is disabled based on electrical current drawn by the voltage converter.

In Example 21, the RF generator of Example 20, wherein the RF output circuit includes a comparator circuit coupled to the voltage converter, and the electrical current and a reference current value are provided to a comparator circuit.

In Example 22, the RF generator of Example 20, comprising a measurement circuit coupled to the RF output circuit and to the controller, wherein the measurement circuit is applied to determine the electrical current and provide a digitized signal to the controller.

In Example 23, the RF generator of Example 19, wherein the electrical power is one of an apparent electrical power or a real electrical power.

In Example 24, the RF generator of Example 19, wherein the electrical power control is asserted via disabling the RF output circuit based on a thermal signal.

In Example 25, the RF generator of Example 24, comprising a thermal sensor connector and wherein the RF puncture device includes a thermal sensor proximate the active electrode.

In Example 26, the RF generator of Example 25, comprising a measurement circuit coupled to the thermal sensor connector and to the controller.

In Example 27, the RF generator of Example 16, wherein the controller is configured to assert the electrical power based on one of a power of the RF puncture signal or a voltage of the RF puncture signal.

In Example 28, the RF generator of any Example 16, wherein the controller is configured to monitor the puncture signal for desiccation based on time of insufficient power of the RF puncture signal.

In Example 29, the RF generator of Example 16, wherein the controller is configured to monitor the puncture signal for fulguration based on time of sufficient power of the RF puncture signal.

In Example 30, the RF generator of Example 16, wherein the RF puncture device is an electrosurgical transseptal guidewire.

In Example 31, a radiofrequency (RF) generator for use in a tissue puncture system for puncturing a tissue in a body, the RF generator comprising: an RF energy source having an active connector and a return connector, the active connector configured to be coupled to an active electrode of an RF puncture device and the return connector configured to be coupled to a return electrode, the active electrode and return electrode configured to couple to the body; and a controller coupled to the RF energy source, the controller configured to: apply an RF puncture signal based on a feedforward control setting, assert an electrical power control of the puncture signal based on thermal energy to the tissue applied with the RF puncture signal from one of a power of the RF puncture signal or a voltage of the RF puncture signal, and monitor the puncture signal for desiccation and fulguration of the tissue based on thermal energy applied to the tissue with the RF puncture signal, wherein the controller is configured to monitor the puncture signal for desiccation based on time of insufficient power of the RF puncture signal, and wherein the controller is configured to monitor the puncture signal for fulguration based on time of sufficient power of the RF puncture signal.

In Example 32, the RF generator of Example 31, comprising an RF output circuit coupled to the controller and wherein the electrical power control is asserted via disabling an RF output circuit

In Example 33, the RF generator of Example 31, wherein the feedforward control setting is based on a nominal feedforward control setting stored in a non-transitory memory device.

In Example 34, a method of use in a radiofrequency (RF) generator in a tissue puncture system, the RF generator including an RF energy source having an active connector and a return connector, the active connector configured to be coupled to an active electrode of an RF puncture device and the return connector configured to be coupled to a return electrode, the active electrode and return electrode configured to couple to a body, the method comprising: applying an RF puncture signal based on a feedforward control setting; asserting an electrical power control of the puncture signal based on thermal energy to the tissue applied with the RF puncture signal; and monitoring the puncture signal for desiccation and fulguration of the tissue based on thermal energy applied to the tissue with the RF puncture signal.

In Example 35, the method of Example 34, wherein the puncture signal is monitored for desiccation based on time of insufficient power of the RF puncture signal for fulguration based on time of sufficient power of the RF puncture signal.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a block diagram illustrating an embodiment of an electrosurgical generator configured for use with the electrosurgical system of FIG. 1.

FIG. 3 is a block diagram illustrating an embodiment of a method of applying an electrical puncture signal using the electrosurgical generator of FIG. 2 in the electrosurgical system of FIG. 1.

FIG. 4A is block diagram illustrating a first embodiment of the electrosurgical generator of FIG. 2.

FIG. 4B is block diagram illustrating a second embodiment of the electrosurgical generator of FIG. 2.

FIG. 4C is block diagram illustrating a third embodiment of the electrosurgical generator of FIG. 2.

FIG. 5 is a flow chart illustrating a first embodiment of a method of a feature of the method of FIG. 3.

FIG. 6 is a flow chart illustrating a second embodiment of a method of a feature of the method of FIG. 3.

FIG. 7 is a flow chart illustrating a third embodiment of a method of a feature of the method of FIG. 3.

FIG. 8A is a graph of plots of real electrical power and apparent electrical power with respect to time during a medical procedure.

FIG. 8B is a graph of plots of electrical current magnitude and voltage magnitude with respect to time.

FIG. 9 is a flow chart illustrating another embodiment of a method of FIG. 3.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Tissue punctures via radiofrequency (RF) energy are associated with thermal energy transfer between a puncture electrode and target tissue. Thermal energy transfer factors used in evaluating effective tissue puncture include temperature at an interface between the tissue and electrode and thermal ramp rates, or how quickly the interface can get to the desired temperature. The desired temperature or temperature region of the tissue-electrode interface for a tissue crossing preferably achieves tissue vaporization in which water inside of the tissue cells flashes to steam and bursts the cells. In one instance, an interface temperature that causes vaporization also encapsulates the electrode with an electrically insulative bubble in the blood pool after puncture, which facilitates a transition to a desirable low current delivery of an energized electrode. Tissue fulguration occurs if too much thermal energy is delivered to the interface, and tissue fulguration can include aggressive heating of blood and damage to tissue outside of the target region. In one instance, an electrode temperature that causes fulguration does not effectively encapsulate the electrode with an electrically insulative bubble in the blood pool after puncture, which compounds ill effects of fulguration. In one example, too high of a thermal ramp rate can cause the interface to overshoot its target temperature, which leads to tissue fulguration. On the other hand, tissue desiccation occurs if not enough thermal energy is delivered to the interface, and desiccation results in dried tissue that forms a scar that is difficult to cross. Crossing desiccated tissue can cause collateral damage to surrounding tissue. In one example, too low of a thermal ramp rate can cause the interface to spend too much time with insufficient thermal energy, which can lead to tissue desiccation.

Electrosurgical generators include circuits to provide RF energy sources to deliver RF energy to electrosurgical devices to produce tissue punctures. To achieve rapid heating to target temperatures, electrosurgical generators can leverage voltage-feedback control to target voltages that are known to produce desirable temperatures at the puncture electrodes. A standard Proportional-Integral-Derivative (PID) controller, which applies a closed-loop feedback mechanism, ramps voltages too slowly to achieve a desirable puncture. An electrosurgical generator of the disclosure applies a feedforward mechanism using a feedforward control setting to achieve rapid heating to target temperatures. The feedforward mechanism generally avoids insufficient energy at ramp up and the likelihood of tissue desiccation but leads to a possibility of causing fulguration if the temperature becomes too high at the interface. The electrosurgical generator clamps thermal energy delivered to the electrosurgical device, such as via thermal measurement hardware, electrical energy measurement hardware, or both. Limiting thermal energy in such a fashion allows for aggressive feedforward control that also reduces the likelihood of the tissue-electrode interface from transitioning to a tissue fulguration temperature region. The electrosurgical generator further monitors RF energy delivery parameters to detect whether the temperatures at the tissue-electrode interface transition to the desiccation or tissue fulguration temperature regions. The disclosure is related to an electrosurgical generator, electrosurgical systems, and methods to reduce the likelihood of tissue fulguration and tissue desiccation after a rapid ramp up in voltage.

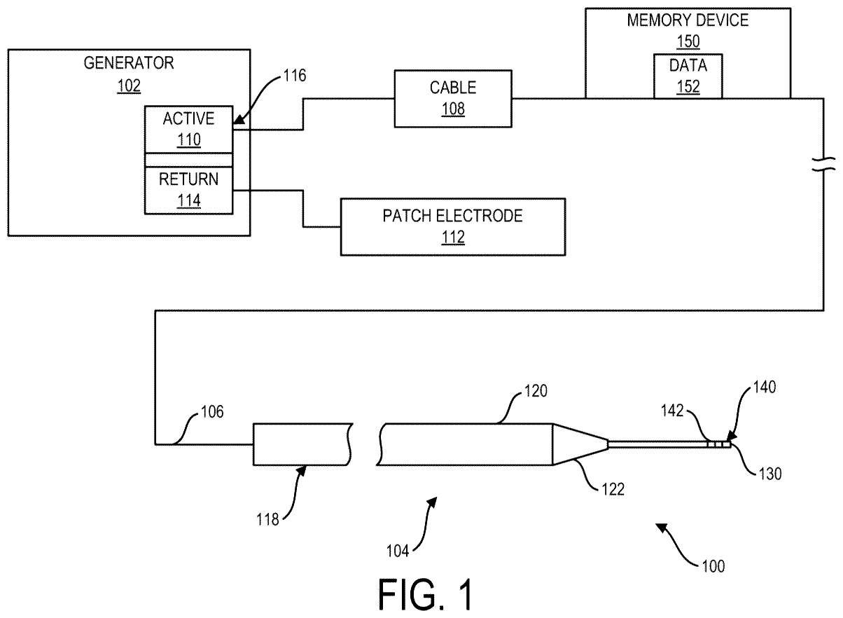

FIG. 1 illustrates an embodiment of an electrosurgical system 100 to facilitate vascular access to a heart and provide catheter positioning within cardiac anatomy. The embodiment of the medical system 100 includes an electrosurgical unit, such as an electrosurgical generator 102 and an electrosurgical device, such as an electrosurgical puncture device 104. In one embodiment, the electrosurgical puncture device 104 includes an electrosurgical transseptal guidewire 106. In the illustration, the electrosurgical transseptal guidewire 106 is electrically coupled to the electrosurgical generator 102 via cable 108, such as to an active connector 110 on the electrosurgical generator 102. The electrosurgical generator 102 is configured to provide a source of energy, such as RF energy to the electrosurgical transseptal guidewire 106 via the cable 108. In some embodiments, the system 100 includes a ground pad electrode, or indifferent (dispersive) patch electrode 112 electrically coupled to the electrosurgical generator 102, such as to a return connector 114 on the electrosurgical generator 102, for use with the electrosurgical transseptal guidewire 106 in a monopolar configuration. In some embodiments, the electrosurgical transseptal guidewire is implemented in a bipolar configuration using a pair of electrodes on the guidewire and without a separate patch electrode. The active connector 110 and return connector 114 are included as a set of receptacles 116 on the generator 102.

The electrosurgical generator 102 is configured to provide the source of RF energy to the electrosurgical transseptal guidewire 106 for a puncture operation with the electrosurgical device 104. In some embodiments, the electrosurgical generator 102 includes an interface including a set of user accessible controls, device connectors such as receptacles 116, and an output device such as a display device, speakers, and lights. During a monopolar puncture operation of electrosurgical generator 102, a first electrode, often referred to as the active electrode, is provided with the electrosurgical device 104 in general and with the transseptal guidewire 106 in the illustration while a second electrode, such as the patch electrode 112, is typically located on the back, buttocks, upper leg, or other suitable anatomical location of the patient during surgery. In such a configuration, the patch electrode 112 is often referred to as a patient return electrode. An electrical circuit of RF energy is formed between the active electrode and the patch electrode 112 through the patient as a patient circuit, and an RF puncture signal through the patient circuit is used to puncture tissue at the active electrode. For example, RF energy for a puncture function in a monopolar mode may be provided at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 106. In one example, the electrosurgical generator 102 provides RF energy to the active electrode as an alternating current having a frequency in the range of 100 kHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected electrosurgical device 104. The actual pulses are often sinusoidal or square waves and bi-phasic, that is alternating positive and negative amplitudes.

The illustrated electrosurgical puncture device 104 includes the electrosurgical transseptal guidewire 106 and a delivery component 118. While embodiments of the disclosure are described with reference to punctures in tissue with a transseptal guidewire for illustration, the features of the disclosure can be used with other electrosurgical devices. The delivery component 118 includes an elongated shaft 120 having a shaft distal tip and defines a longitudinally extending axial shaft lumen. The electrosurgical transseptal guidewire 106 is adapted to be disposed within the shaft lumen and electrically coupled to the RF energy source at the active connector 110. In some embodiments, the delivery component 118 can include an elongate sheath, and the electrosurgical transseptal guidewire 106 is disposed within the sheath. In another embodiment, the delivery component 118 can include a dilator/sheath assembly, and the electrosurgical transseptal guidewire 106 is disposed within the dilator/sheath assembly. For instance, the elongated shaft 120 includes a distal tapered portion 122 with an enlargement of cross-sectional area with respect to the shaft distal tip. As the distal tapered portion 122 is passed through an aperture from the shaft distal tip, the enlargement of cross-sectional area dilates the aperture. The dilator can be configured as a straight dilator, as illustrated, or a curved dilator. The elongated shaft 120 can be made from various materials including insulative materials such as high-density polyethylene (HDPE). The shaft 120 and distal tapered portion 122 can include various materials such as metal hypotubes as well.

The electrosurgical transseptal guidewire 106 includes a puncture wire shaft with a puncture wire proximal portion and a puncture wire distal portion having a puncture wire distal tip 130. The puncture wire distal tip 130 includes a puncture electrode 140 adapted to deliver the RF energy. The puncture electrode 140 is configured as the active electrode. The puncture wire proximal portion includes an end connector configured to electrically couple to cable 108 and receive an RF signal from the electrosurgical generator 102. In one example, the electrosurgical transeptal guidewire 106 can be coupled to and uncoupled from the cable 108 depending on whether the electrosurgical transeptal guidewire 106 is used as an electrosurgical puncture device or as an exchange rail, for instance. The transseptal guidewire 106 is configured to conduct the RF signal from the proximal portion along the puncture wire shaft to the electrode 140. In some embodiments, the puncture wire shaft is constructed from an electrically conductive material having an electrically insulative outer coating. In some embodiments, the electrically conductive material is a flexible, shape memory material such as a nickel titanium alloy or nitinol. The exposed electrode 140 is configured to apply the RF energy, such as to puncture tissue.

In some embodiments, the electrosurgical transseptal guidewire 106 includes a thermal sensor 142 proximate the puncture electrode 140, such as disposed on the puncture wire shaft. The thermal sensor 142 is configured to detect a temperature or temperature variation and provide a response such as an electrical signal. In one embodiment, the thermal sensor 142 includes a thermal couple or other sensor having relatively fast reaction times, such as in the millisecond range, to allow the electrosurgical generator 102 to determine thermal trends at the electrode-tissue interface and process a signal from the thermal sensor 142 in a feedback control scheme. The thermal sensor 142 is coupled to a lead along the puncture wire shaft and typically electrically insulated from the electrical conductor coupled to the puncture electrode. The puncture wire proximal portion includes a thermal sensor end connector configured to couple to the cable 108. Some embodiments of the electrosurgical generator 102 include a connection for the thermal sensor end connector in receptacles 116. The electrosurgical generator 102 is configured to receive and process the signal from the thermal sensor.

In the illustrated example, the electrosurgical transseptal guidewire 106 is configured as a multifunction conductive guidewire. For instance, the transseptal guidewire 106 can be used, without exchanges, as a guidewire, a transseptal puncture device, and as an exchange rail for delivering therapy sheaths. Such embodiments provide efficiencies to medical procedures as the transseptal guidewire 106 performs multiple functions and reduces the amount of device exchanges in the medical procedure. The distal tip 130 of the transseptal guidewire 106 is extendable from the distal end of the delivery component 118 such that the delivery component 118 is retractable from the patient over the guidewire 106 while the guidewire distal tip 130 is disposed within the heart. The transseptal guidewire 106 is sufficiently thin and flexible to access the various chambers of the heart. The electrode 140 on the transseptal guidewire 106 is operable to deliver RF energy to puncture the atrial septum from the right atrium, and the distal portion of the transseptal guidewire can be advanced through an aperture in the septum formed by the puncture. Once advanced through the aperture and sufficiently extended from within the delivery component 118, the distal portion of the transseptal guidewire 106 is biased to form a coil for anchoring the distal end beyond the aperture. The delivery component 118 is retractable from the patient over the anchored transseptal guidewire 106. The transseptal guidewire 106 can also support the installation of therapy devices to a therapy location in the patient's heart, such as tubular members or other catheters and for advancing other devices within the heart.

In some embodiments, the electrosurgical generator 102 is automatically programmed for operation based on the particular configuration of the electrosurgical device 104. The transseptal guidewire 106 of the illustrated embodiment includes a memory device 150 (non-transitory memory) storing a set of parameters 152 associated with the design and configuration of the transseptal guidewire 106. The memory device 150 is coupled to the electrosurgical generator 102 via the receptacles 116, such as a serial interface, and the electrosurgical generator 102 is configured to read the parameters 152 to program the controls to be suited for the associated transseptal guidewire 106. The memory device 150 can store the parameters 152 in various memory segments having lookup tables or other data structures to provide data to be loaded into a memory device in the electrosurgical generator 102 and read by a controller of the electrosurgical generator to affect operation. Example parameters 152 can include model number of the transseptal guidewire 106, acceptable voltages or power levels for use with the transseptal device 106, whether the transseptal device 106 is configured for single use or multiple uses, impedance of the transseptal device 106, as well as other parameters. In some embodiments, the electrosurgical generator 102 can be programmed to write to memory segments on the memory device 150 as well as to read the memory device 150.

In an anticipated use of the system 100, the electrosurgical device 104 is coupled to the electrosurgical generator 102, and if the electrosurgical device 104 is to be configured in a monopolar mode, the patch electrode 112 is coupled to the patient. The electrosurgical generator 102 can be set to a puncture mode, such as an energy output of approximately 10 watts. In some examples, femoral access is obtained via a conventional percutaneous needle, and the transseptal guidewire 106 is inserted into the vasculature and advanced to the superior vena cava. The distal tip of the delivery component 118 is advanced over the proximal portion of the transseptal guidewire 106, to the superior vena cava. Under visualization, the distal tapered portion 122 is moved from the superior vena cava to the right atrial septum and then to the fossa ovalis of the heart. Once the delivery component distal tip is confirmed at the fossa ovalis, the electrode 140 of the transseptal guidewire 106 is advanced from the distal tip of the delivery component 118. In one example, the exposed puncture electrode 140 of the transseptal guidewire 106 is extended a few millimeters from the distal tip of the delivery component 118 to tent the heart tissue, and the transseptal guidewire 106 can be locked in position with respect to the delivery component 118. Forward pressure is applied to the electrosurgical device 104 and the transseptal guidewire 106 is actuated to apply the RF energy to the electrode 140 and puncture the fossa ovalis. The RF energy punctures the fossa ovalis and creates the aperture. The transseptal guidewire 108 is unlocked from the delivery component 118, and the transseptal guidewire 106 is extended through the aperture. In the embodiment of the delivery component 118 configured as the dilator/sheath assembly, the distal tapered portion 122 of a dilator is advanced into the puncture site to expand the aperture in the septum. The delivery component 118 can be retracted from the patient over the transseptal guidewire 106, and transseptal guidewire 106 can provide support for the installation of tubular members or other catheters and for advancing other devices within the heart.

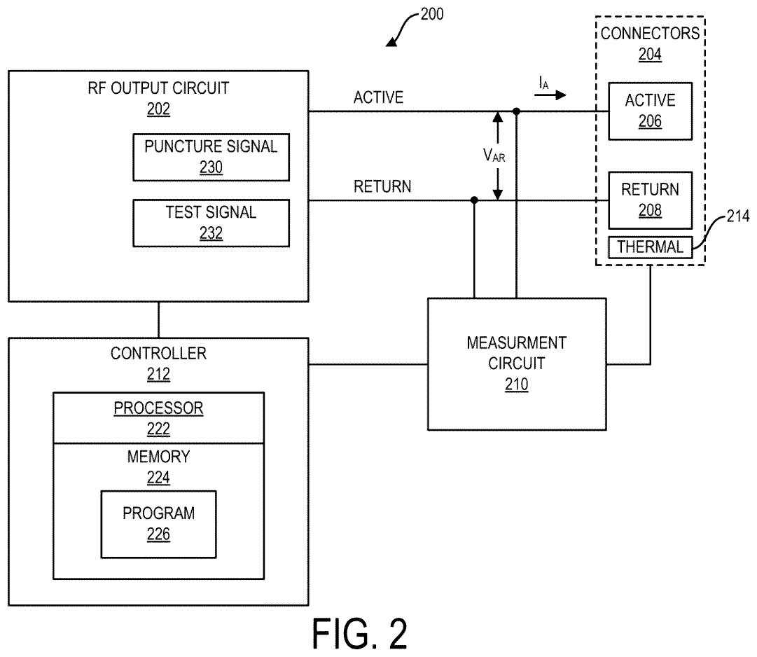

FIG. 2 illustrates an embodiment of electrosurgical unit, such as an electrosurgical generator 200, which can correspond with electrosurgical generator 102 in system 100. The electrosurgical unit or RF generator is described with reference to transseptal punctures in this disclosure for illustration only. But the principles of the electrosurgical generator of this disclosure are not limited to transseptal punctures. For instance, the RF generator 200 can be used in other medical systems including systems for ablation and electrocautery. The electrosurgical generator 200 is configured to generate an RF puncture signal based on a feedforward control setting. The feedforward control setting generates a rapid ramp up in thermal energy from the RF puncture signal that passes through the tissue desiccation temperature region relatively quickly and into the tissue vaporization temperature region. In embodiments, the electrosurgical generator 200 is also configured to implement a control scheme to generally maintain the RF puncture signal to produce a thermal energy in the tissue vaporization region and away from thermal energy regions that cause tissue desiccation and tissue fulguration. The control scheme monitors parameters of the RF puncture delivery to avoid the tissue desiccation and tissue fulguration regions.

The electrosurgical generator 200 includes an RF output circuit 202, a plurality of device connectors 204 including an active connector 206, a return connector 208, and (in some embodiments) a thermal sensor connector 214, a measurement circuit 210, and a controller 212. The RF output circuit 202 is configured to generate an RF puncture signal. In some embodiments, the RF output circuit 202 generates the puncture signal based on feedforward settings determined from the controller 212 and applies closed-loop feedback, such as via a PID controller, during delivery of the puncture signal to control the output of the puncture signal. The RF output circuit 202 can include a power supply to provide a direct current (DC) supply voltage signal to an RF voltage converter that converts the DC signal to an alternating current (AC) signal as an RF voltage. The RF voltage is provided to a waveform generator of the RF output circuit 202, which is configured to generate a plurality of voltages, waveforms having various duty cycles, peak voltages, crest factors, frequencies, and other suitable parameters. The RF output circuit provides the puncture signal to the active connector 206.

The device connectors 204 can be configured to include receptacles located on a housing of the RF generator 200 that can be mechanically coupled to electrosurgical devices. The device connectors 204 are configured to electrically couple the electrosurgical generator 200 to various electrosurgical devices. In some embodiments, the device connectors 204 correspond with the receptacles 116 of FIG. 1. In some embodiments, the active connector 206 is suitable for electrically coupling to cable 108, which can be electrically coupled to the transseptal guidewire 106. The return connector 208 is suitable for electrically coupling to the ground pad electrode 112 when an electrosurgical device is operated in a monopolar mode (or to a return electrode on the electrosurgical device when operated in a bipolar mode). During a medical procedure, the electrode 140 of the transseptal guidewire 106 and the return electrode, such as the ground pad electrode 112, are coupled to the patient, such heart tissue and the torso, respectively, to form a patient circuit with the RF output circuit 202 for the puncture signal. In embodiments of the electrosurgical generator 200 configured to operate with an electrosurgical device 104 having a thermal sensor, such as thermal sensor 142, the device connectors 204 include a thermal sensor connector 214, which is configured to be coupled to the thermal sensor end connector and receive a thermal signal from the thermal sensor.

The measurement circuit 210 is electrically coupled to the device connectors 204 and, in some embodiments, is configured to determine current and voltage measurements or impedance measurements from an excitation waveform resulting from the puncture signal generated by the RF output circuit 202 in the patient circuit and present the current and voltage or impedance measurements to the controller 212. The measurement circuit 210 can include circuit elements or paths electrically coupled to the RF output circuit 202 or at least some of the output connectors 204 including the active connector 206 and return connector 208 and is configured to provide a signal representative of the active and return voltages and active current. The circuit elements can include current probes to measure currents of interest. In one embodiment, the measurement circuit 210 includes an analog to digital converter coupled to the circuit elements and to the controller 212 to provide digital signals to the controller 212. In some embodiments, the measurement circuit 210 is coupled to the thermal sensor connector 214 and configured to receive the thermal sensor signal. The measurement circuit 210 coupled to the thermal sensor connector 214 is configured to provide analog signal conditioning and digitization of the thermal sensor signal. In some embodiments the measurement circuit 210 is configured to receive both the puncture signal and the thermal sensor signal. In other embodiments, the measurement circuit 210 is configured to receive one of the puncture signal or the thermal sensor signal.

The controller 212 is coupled to the output circuit 202 and is configured to control, such as to adjust settings and cause operation of, the output circuit 202. The controller 212 in embodiments includes a processor 222 operably connected to a memory device 224 (non-transitory memory). The memory device 224 can store processor executable instructions configured to control the processor, such as a program 226. Examples of a memory device 224 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. In some embodiments, the memory device 224 includes a non-volatile component for storing tables or other data structures used with the program 226. The processor 222 includes an output port that allows the processor 222 to control the output of the controller 212 according to a programmed scheme.

In other embodiments, the functionalities of controller 212 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, of this disclosure illustrated with the processor 222, memory 224, and program 226 can be implemented in such electronic circuitry.

The measurement circuit 210 receives the resultant puncture signal and, in one embodiment, converts the analog signal to a digital signal for the controller 212 to process. The measurement circuit 210 is electrically coupled to the active and return connectors 206, 208 and determines electrical current waveforms and voltage waveforms of the puncture signal, such as current at the active connector 206, Iₐ, and voltage across the active connector 206 and return connector 208, Vₐᵣ, performs an analog to digital conversion of the determination, and provides the digitized measurement to the controller 212. Using the current and voltage measurements from the measurement circuit 210, the controller 212 can determine impedance and apparent power. By determining phase information with the measurement circuit 210, the control can determine real power and complex impedance. For instance, impedance of the patient circuit Zₚₐₜᵢₑₙₜ is determined via (Vₐᵣ)/(Iₐ). Apparent power Pₐₚₚₐᵣₑₙₜ is determined via the root-mean square value of voltage in volts multiplied by the root-mean-square value of current in amperes, or (Vₐᵣ)*(Iₐ). Real power in AC signals Pᵣₑₐₗ is determined via the root-mean square value of voltage in volts multiplied by the root-mean-square value of current in amperes multiplied via a cosine of the phase angle θ of which the voltage sine wave leads the current sine wave, i.e., (θᵥ-θᵢ), or (Vₐᵣ)*(Iₐ)cos θ. In another embodiment, real power is determined by one-half the peak voltage in volts multiplied by the peak current in amperes multiplied via the cosine of the phase angle θ. In one embodiment, the measurement circuit 210 determines both the voltage Vₐᵣ and current Iₐ to determine both the magnitude and phase angle for the complex impedance of the electrical load coupled to the active and return connectors 206, 208. In one embodiment, the controller 212 determines complex impedance measurements via a Fourier transform technique on the voltage Vₐᵣ and current Iₐ data of the measured test signal. In other embodiments, the complex impedance is determined via under-sampling/over-sampling the voltage Vₐᵣ and current Iₐ to create a harmonic equivalent of the resultant waveform of the test signal and performing a sum-of-least-squares calculation to measure root-mean-square equivalents of the waveform, which is used to calculate impedance. In still another embodiment, hardware is applied to convert the voltage Vₐᵣ and current Iₐ to equivalent direct current signals, which is used to extract phase information. Other mechanisms and processes can be applied to determine complex impedance.

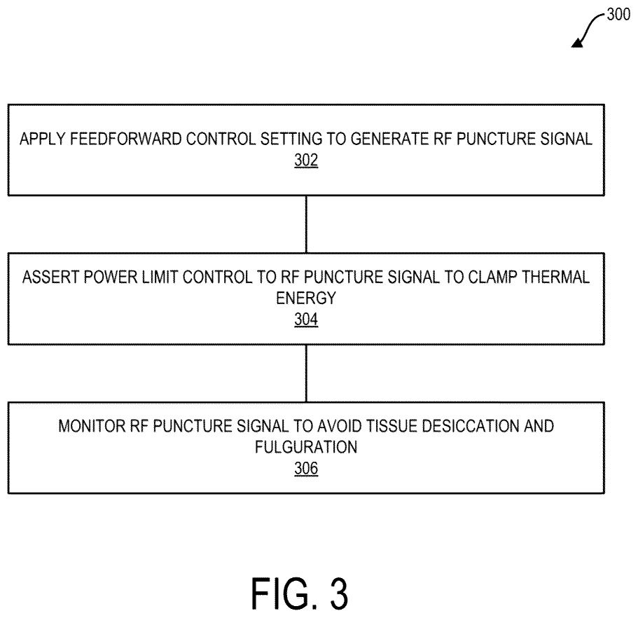

FIG. 3 illustrates an embodiment of a method 300 to perform an RF puncture delivery providing thermal energy maintained in a tissue vaporization region. In embodiments, the method 300 is implemented with the electrosurgical generator 200, such as via one or more of electrical circuitry, logic devices, and a set of executable instructions in program 226 of the controller 212. The electrosurgical generator 200 provides the RF energy source and the active connector 206 is configured to be coupled to an active electrode of an RF puncture device and the return connector 208 is configured to be coupled to a return electrode. The active electrode and return electrode are configured to be coupled to the body, such as the heart tissue and torso, respectively, and form a patient circuit. The method 300 generates an RF puncture signal based on a feedforward control setting in a feedforward mechanism to achieve rapid heating to target temperatures at 302. The feedforward control signal is applied to generate the RF puncture signal. The feedforward control setting generates a rapid ramp up in thermal energy from the RF puncture signal that passes through the tissue desiccation temperature region relatively quickly and into the tissue vaporization temperature region. The method 300 clamps thermal energy delivered to the electrosurgical device by asserting a power limit control at 304. The power limit control allows the feedforward mechanism to be aggressive and to transition into the vaporization temperature region quickly without transitioning into a fulguration temperature region. In embodiments, the electrosurgical generator employs one or more of circuitry, logic and software to monitor at least one of thermal energy delivered to the electrosurgical device or electrical energy delivered to the patient circuit to determine whether to limit power to the RF output circuit 202. In some embodiments, the thermal energy or electrical energy are clamped at a predetermined instantaneous value. The system monitors a temperature signal or an energy determination from electrical signal characteristics such as voltage and current and stops the delivery of the puncture signal in response to the temperature signal or energy determination reaching or exceeding a threshold value. In another embodiment, the thermal energy or electrical energy are clamped at a rolling integral value. In some embodiments, an amount of thermal energy is determined from the RF puncture signal. The method 300 monitors parameters of the RF puncture signal to avoid the tissue desiccation and tissue fulguration regions at 306. In some embodiments, the monitoring at 306 and power limiting at 304 are performed concurrently. In other examples, the monitoring is performed to detect an abnormal power delivery configuration. In one embodiment, tissue desiccation is determined from an amount of time spent without sufficient power in the RF puncture signal. In another embodiment, tissue fulguration is determined from an amount of time spent with sufficient power in the RF puncture signal.

In the electrosurgical generator 200 of FIG. 2, an RF energy source, such as RF output circuit 202, applies a puncture signal via feedforward mechanism at 302. In embodiments, a feedforward control setting is determined based on a nominal feedforward control setting is stored in memory 224. By way of example, the electrosurgical generator 200 uses a control mechanism or circuit to set to the voltage level delivered to the patient. For instance, the electrosurgical generator 200 can use a controllable digital-to-analog (DAC) converter to set the feedforward volage (V_{FF}) delivered to the device connectors 204 from the RF output circuit 202. In some embodiments, the DAC receives a signal based on a control setting from the controller of a generator and applies the control setting to adjust an output voltage. In a typical example, the control setting applied to the DAC, or DAC_{SETTING}, to create an output voltage from a feedforward voltage setpoint (V_{FF}) can be determined from a linear relation such as:

DAC_{SETTING,}=V_{FF}*m+b

In the electrosurgical generator 200, the feedforward voltage setpoint V_{FF} in one embodiment is the nominal feedforward voltage value, such as the feedforward voltage expected to be obtained via a nominal feedforward control setting. The feedforward control setting is applied to the RF output circuit 202 to generate the puncture signal.

In some embodiments, the feedforward control setting can be adjusted from a nominal setting or selected from a plurality of available of feedforward control setting based on measurements of electrical loads, type of electrosurgical device coupled to the electrosurgical generator, or other conditions. Feedforward control settings can be adjusted via runtime calculations or from a set of available settings stored in a memory structure such as an array or lookup table of memory device 224. Examples are disclosed in concurrently filed application to Steven Kinio, et al. titled SMART FEEDFORWARD FOR OPTIMAL RF DELIVERY DURING RF PUNCTURE, U.S. Patent Application Ser. No. 63/752,559, filed Jan. 31, 2025, to the present assignee, and incorporated by reference herein to the extent it is not inconsistent with the present disclosure.

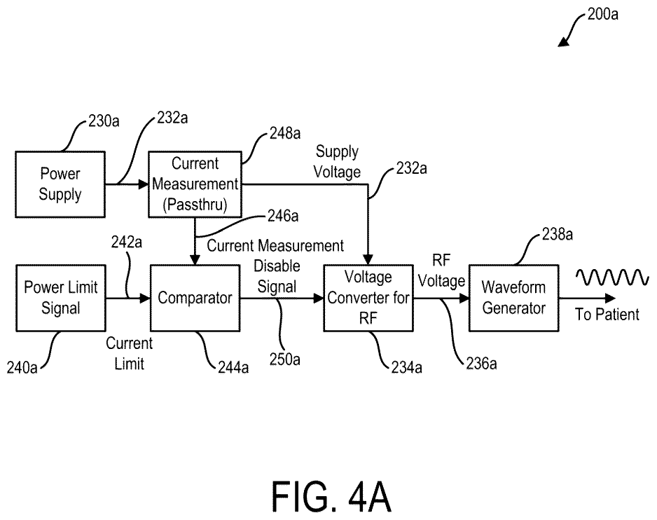

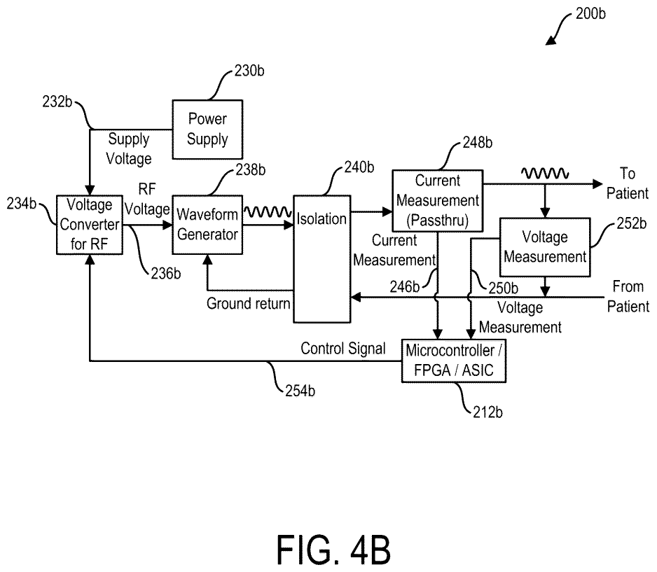

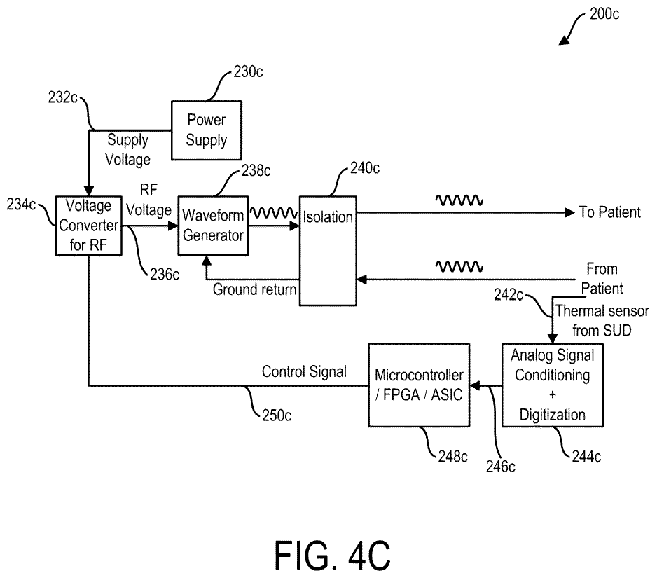

FIG. 4A illustrates features of a first embodiment electrosurgical unit 200a, which is an implementation of electrosurgical generator 200, that applies a control scheme to clamp thermal energy delivered to an electrosurgical device by asserting a power limit control at 304 of method 300. The electrosurgical generator 200a includes a power supply 230a to provide a DC supply voltage electrical signal 232a to an RF voltage converter 234a. The RF voltage converter 234a converts the DC signal to an AC signal as an RF voltage signal 236a. The RF voltage converter 234a provides the RF voltage signal 236a to a waveform generator 238a, which is configured to generate a plurality of voltages, waveforms having various duty cycles, peak voltages, crest factors, frequencies, and other suitable parameters. The waveform generator 238a provides the puncture signal to the active connector 206 and is electrically coupled to the return connector 208.

Electrosurgical generator 200a illustrates electrical components for a hardware implementation of the power limit control assert at 304 of method 300. An embodiment is implemented with a power limit generator 240a providing a current limit signal 242a to a comparator 244a and a current measurement signal 246a provided from a current measurement circuit 248a coupled to the power supply 230a and configured to receive the supply voltage signal 232a, such as a passthrough circuit between the power supply 230a and the RF voltage converter 234a. The current measurement signal 246a is indicative of an amount of electrical current in the DC supply voltage electrical signal 232a. In the illustrated embodiment, the comparator 242a monitors the current drawn by the RF voltage converter 234a and temporarily disables or shuts down the RF voltage converter 234a if it draws a current that exceeds a threshold. The comparator 244a receives the current measurement signal 246a and compares it to the current limit signal 242a, which is indicative of a threshold amount of current for the supply voltage electrical signal 232a. If the current measurement signal 246a indicates the current in the supply voltage electrical signal 232a has surpassed the threshold limit as indicated in the current limit signal 242a, the comparator 244a provides a disable signal 250a to the RF voltage converter. In response to receiving the disable signal 250a, the RF voltage converter 234a will cease or disable operation, such as cease operation until it is reset to impose a duty cycle.

FIG. 4B illustrates features of a second embodiment electrosurgical unit 200b, which is also an implementation of electrosurgical generator 200. Electrosurgical generator 200b applies software or logic for the power limit control assert at 304 of method 300. The electrosurgical generator 200b includes a power supply 230b to provide a DC supply voltage electrical signal 232b to an RF voltage converter 234b. The RF voltage converter 234b converts the DC signal to an AC signal as an RF voltage signal 236b. The RF voltage converter 234b provides the RF voltage signal 236b to a waveform generator 238b, which is configured to generate a plurality of voltages, waveforms having various duty cycles, peak voltages, crest factors, frequencies, and other suitable parameters. The waveform generator 238b provides the puncture signal to the active connector 206 and is electrically coupled to the return connector 208 such as through an electrical isolation component 240b.

An embodiment of the of the power limit control assertion at 304 is implemented with a current measurement signal 246b from a current measurement circuit 248b and a voltage measurement signal 250b from a volage measurement circuit 252b. The current measurement signal 246b and voltage measurement signal 250b are provided to a controller 212b. The current measurement signal 246b indicates an amount of current in the puncture signal through the patient circuit and the voltage measurement signal 250b indicated an amount of voltage across the active and return connectors 206, 208 from the puncture signal. In one embodiment, the current and voltage measurement circuits 248b, 252b include analog to digital converters coupled to circuit elements to provide digital signals to the controller 212b. The controller 212b provides a control signal 254b to the RF voltage converter 234b. In one embodiment, the controller 212b calculates apparent power delivered to the patient with the puncture signal from the current and voltage measurement signals 246b, 250b and, with control signal 254b, reduces a control setpoint in a feedback mechanism if the apparent power exceeds a limit. Apparent power, or apparent electrical power, is the total amount of electrical power flowing in an AC system determined via multiplying voltage by current without reference to phase angle and is measured in volt-amps (VA). In another embodiment, the controller 212b calculates real power delivered to the patient with the puncture signal from the current and voltage measurement signals 246b, 250b and, with control signal 254b, reduces a control setpoint in a feedback mechanism if the real power exceeds a limit. Real power, or real electrical power, is power consumed by the resistive load and is measured in watts (W).

FIG. 4C illustrates features of a third embodiment electrosurgical unit 200c, which is also an implementation of electrosurgical generator 200. Electrosurgical generator 200c applies the power limit control assert at 304 of method 300 via temperature measurement. The electrosurgical generator 200c includes a power supply 230c to provide a DC supply voltage electrical signal 232c to an RF voltage converter 234c. The RF voltage converter 234c converts the DC signal to an AC signal as an RF voltage signal 236c. The RF voltage converter 234c provides the RF voltage signal 236c to a waveform generator 238c, which is configured to generate a plurality of voltages, waveforms having various duty cycles, peak voltages, crest factors, frequencies, and other suitable parameters. The waveform generator 238c provides the puncture signal to the active connector 206 and is electrically coupled to the return connector 208 such as through an electrical isolation component 240c.

An embodiment of the power limit control assertion at 304 is implemented with a thermal sensor signal 242c provided from a thermal sensor on the electrosurgical device, such as thermal sensor 142, and received at a thermal measurement circuit 244c. For instance, the thermal measurement circuit 244c is coupled to thermal sensor connector of device connectors 204. The thermal sensor signal 242c indicates an amount of thermal energy measured proximate the puncture electrode of the electrosurgical device. In one embodiment, the thermal measurement circuit 244c includes circuit components to condition an analog thermal sensor signal 242c and to digitize the conditioned signal. In the illustrated embodiment, a digitized thermal sensor signal 246c is provided to a controller 248c. In one embodiment, the controller 248c processes the received thermal sensor signal 246c to determine, for instance, temperature or thermal trends and adjust a control signal 250c provided to the RF voltage converter 234c based on thermal feedback in the control scheme. The control signal 250c in one embodiment can adjust a voltage output of the RF voltage converter 234c such as RF voltage signal 236c.

Other implementations of electrosurgical generator 200 are contemplated. For instance, some embodiments can include combination of features from two or more embodiments of the illustrated first, second, and third electrosurgical generators 200a, 200b, 200c. Some embodiments include a measurement circuit having current measurement circuit 248b and voltage measurement circuit 252b to generate current and voltage measurement signals, respectively, provided to the controller and thermal measurement circuit 244c to receive a thermal sensor signal 242c provided from a thermal sensor on the electrosurgical device to generate a digitized thermal sensor signal 246c also provided to the controller. In one embodiment, the controller can base a power limit control assert at 304 on either or both of the current and voltage measurement signals and the thermal measurement signals. In another embodiment, the power limit control assert at 304 can be provided by one or both of hardware features included in generator 200a, and logic or software features included in in electrosurgical generator 200b. Still other combinations of features are contemplated.

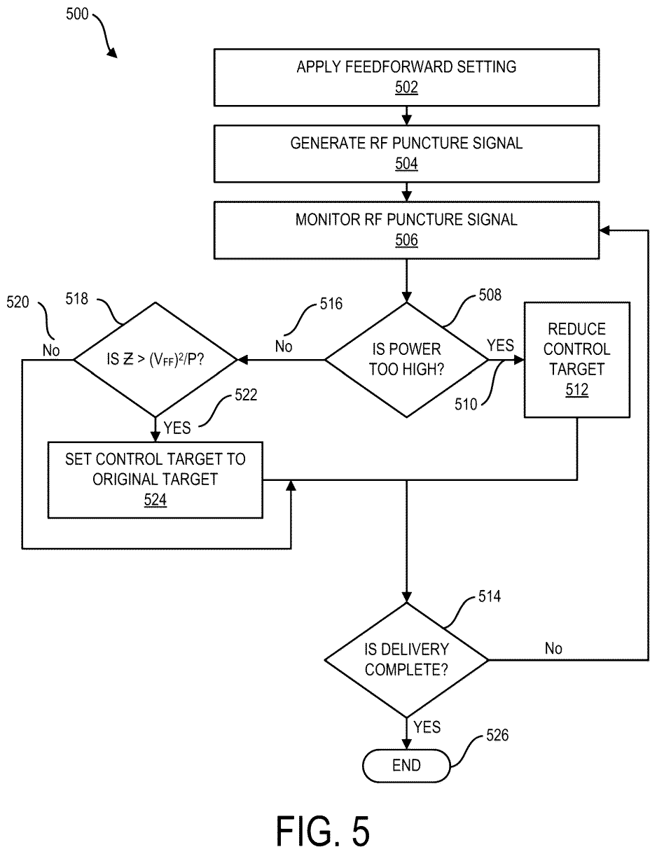

FIG. 5 illustrates a first method 500 to generate an RF puncture signal at 302 and dynamically control the RF output at 304 in method 300 with an electrosurgical generator 200. Method 500 illustrates an embodiment of a feedback control that dynamically limits output of the electrosurgical generator 200 based on determination of real power in the RF puncture signal. A control target matching a desired steady state delivery of the RF puncture signal is selected at 502. The steady state delivery is a steady state voltage, in one embodiment, the steady state voltage is applied as a voltage setpoint. The voltage setpoint corresponds with a feedforward control setting. In one example, the voltage setpoint and corresponding feedforward control setting are stored in the memory device 224 as one or more setpoint-control setting pairs. In one embodiment, the voltage setpoint is the feedforward voltage V_{FF} and the control setting is the control setting applied to the DAC, or DAC_{SETTING}. The control setting is applied, which is now labeled the original control setting, and the RF puncture signal is generated at 504. The puncture signal is monitored, and feedback control of the puncture signal is performed at 506. In one embodiment, the feedback control is performed via a PID controller in the RF output circuit 202.

A determination is made as to whether the real power of the RF puncture signal is above a threshold amount at 508. For example, the measurement circuit 210 provides voltage and current measurements to the controller 212, which determines the real power value from the voltage and current measurements and compares the real power value to a threshold value. If the real power of the RF puncture signal is above the threshold at 510, a control target is reduced to provide a present control target at 512. A determination is made as to whether the delivery of the RF puncture signal is complete at 514. If delivery continues, the puncture signal is monitored, and feedback control of the puncture signal is performed at a return to 506.

If the real power of the RF puncture is below the threshold at 516, a determination is made as to whether the impedance in the patient circuit is greater than a power limit of the RF output circuit 202 divided by the square of the voltage setpoint at 518, or:

Zₚₐₜᵢₑₙₜ>(V_{FF})²/Pₗᵢₘᵢₜ

in which Pₗᵢₘᵢₜ is a determined threshold value of a maximum power output of the RF output circuit 202. If the impedance is not greater at 520, a determination is made as to whether the delivery of the RF puncture signal is complete at 514. If the impedance is greater at 522, the present control target is reset to the original control target at 524, and a determination is made as to whether the delivery of the RF puncture signal is complete at 514. If delivery continues, the puncture signal is monitored, and feedback control of the puncture signal is performed at a return to 506; otherwise, the first method is ended at 526.

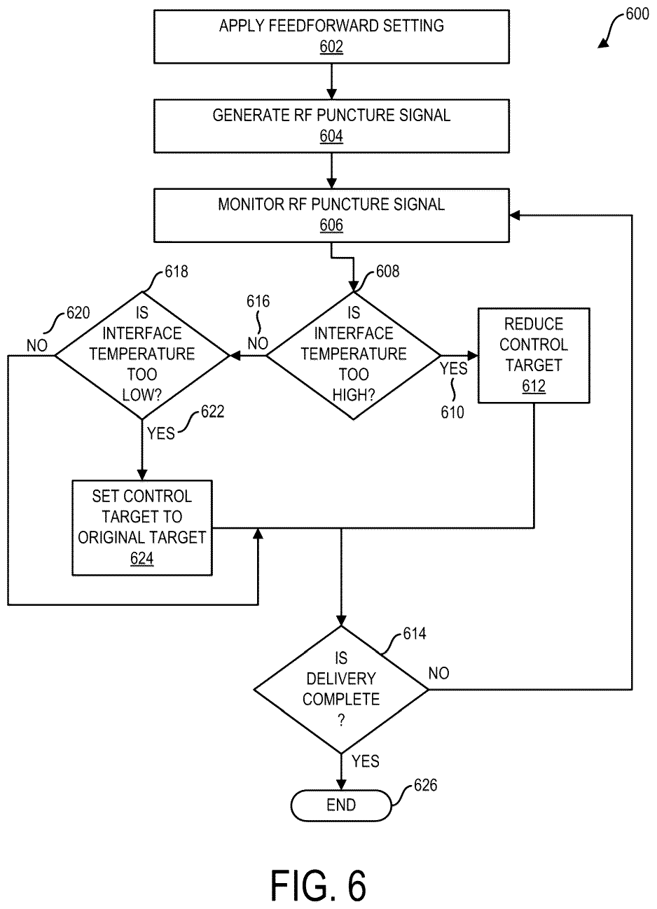

FIG. 6 illustrates a second method 600 to generate an RF puncture signal at 302 and dynamically control the RF output at 304 in method 300 with an electrosurgical generator 200. Method 600 illustrates an embodiment of a feedback control that dynamically limits output of the electrosurgical generator 200 based on temperature measured at the thermal sensor 142. A control setting matching a desired steady state delivery of the RF puncture signal is selected at 602. The steady state delivery is a steady state voltage, in one embodiment, the steady state voltage is applied as a voltage setpoint. The voltage setpoint corresponds with a feedforward control setting. The control setting is applied, which is now labeled the original control setting, and the RF puncture signal is generated at 604. The puncture signal is monitored, and feedback control of the puncture signal is performed at 606.

A determination is made as to whether the measured temperature is above a threshold amount at 608. For example, measurement circuit provides a digitized thermal sensor signal to the controller, which compares the representative temperature to a threshold value. If the measured temperature is above the threshold at 610, the control target is reduced to provide a present control target at 612. A determination is made as to whether the delivery of the RF puncture signal is complete at 614. If delivery continues, the puncture signal is monitored, and feedback control of the puncture signal is performed at a return to 606.

If the temperature is determined not to be above the threshold at 616, a determination is made as to whether the temperature is too low, compared to a lower limit temperature at 618. If the temperature is not below the lower limit temperature, at 620, a determination is made as to whether the delivery of the RF puncture signal is complete at 614. If the temperature is below the lower limit temperature at 622, the present control target is increased, such as incremented, and becomes the present control target at 624, and a determination is made as to whether the delivery of the RF puncture signal is complete at 614. If delivery continues, the puncture signal is monitored, and feedback control of the puncture signal is performed at a return to 606; otherwise, the second method 700 is ended at 626.

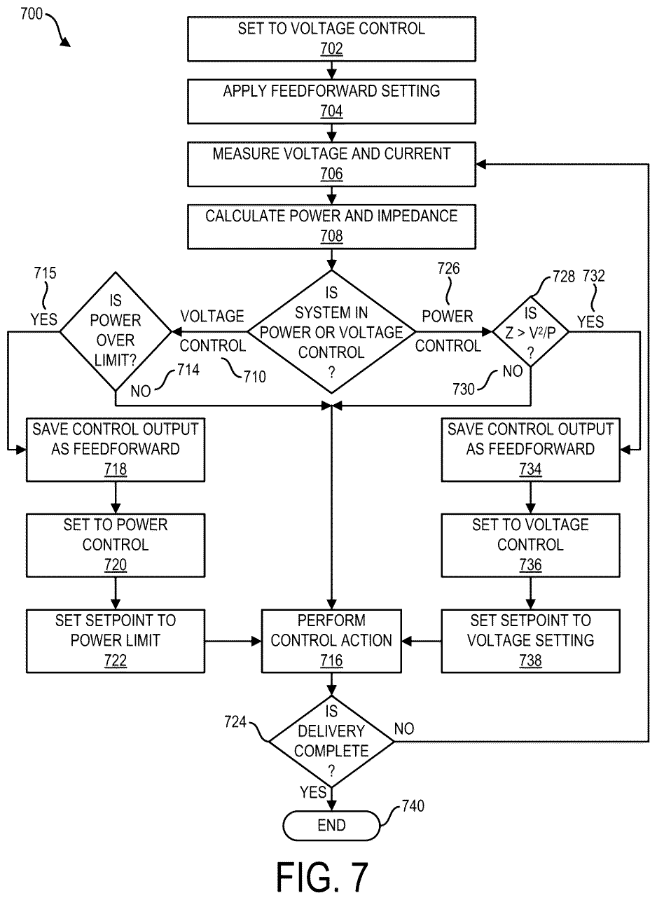

FIG. 7 illustrates a third method 700 to generate an RF puncture signal at 302 and dynamically control the RF output of 302, 304 in method 300 with a hybrid type of electrosurgical generator 200. An embodiment of the hybrid type of electrosurgical generator 200 for use with method 700 is configurable in both a power control mode and a voltage control mode. In the power control mode, the controller 212 makes determinations regarding an electrical power to manage the RF puncture signal. In the voltage control mode, the controller 212 makes determinations regarding voltage to manage the RF puncture signal. In the embodiment of the electrosurgical generator 200, the controller 212 is capable of operating in one or the other of power control mode and voltage control mode. The feedforward mechanism of the controller 212 in voltage control mode applies a feedforward voltage setpoint, which generates an effective thermal ramp rate in generating the RF puncture signal. If the power in the RF puncture signal exceeds a power limit (after a debounce or settling period applied to discern noise and artifacts in the measured RF puncture signal), the controller 212 switches to power control mode to target power limiting of the RF puncture signal. To create a relatively efficient change from voltage control mode to power control mode, the last command from the controller in voltage control mode before the change in mode is provided as a feedforward command in power control mode. While in power control mode, the controller 212 monitors impedance in the patient circuit. If the impedance of the patient circuit is such that:

Zₚₐₜᵢₑₙₜ>(Vₐᵣ)²/Pₗᵢₘᵢₜ

(after a debounce or settling period applied to discern noise and artifacts in the measured RF puncture signal), the controller 212 can switch from power control mode back to voltage control mode. To create a relatively efficient change from power control mode to voltage control mode, the last command from the controller 212 in power control mode before the change in mode is provided as a feedforward command in voltage control mode. In one embodiment, accumulated errors or adjustment values are reset in each change between modes to reduce or prevent control actions from jumping. In one example, changing from voltage control mode to power control mode resets the accumulated units of error in voltage control mode to zero units such that a subsequent change from power control mode to voltage control mode will result in beginning voltage control mode in zero units of accumulated error. Tuning of voltage control mode and power control mode can apply different PID gains because the setpoints are different, but, in some embodiments, both feedback systems are regulated by modifying voltage delivered to the patient in the RF puncture signal.

Method 700 begins with the controller 212 in voltage control mode at 702. A control setting matching a desired steady state delivery of the RF puncture signal is selected at 704, and the RF puncture signal is generated. Current and voltage of the RF puncture signal is measured at 706, such as by the measurement circuit 210; and impedance of the patient circuit and apparent power of the RF signal is determined at 708, such as by the controller 212.

If the controller 212 is in voltage control mode at 710, a determination is made as to whether the apparent power of the RF puncture signal is above a threshold amount at 712. For example, the measurement circuit 210 provides voltage and current measurements to the controller 212, which determines the apparent power value from the voltage and current measurements and compares the apparent power value to a threshold value, or Pₗᵢₘᵢₜ in the embodiment (after the debounce or settling period). If the apparent power of the RF puncture signal is below the threshold at 714, the control performs a control action on the RF puncture signal at 716, and therapy continues in voltage control mode. If the apparent power of the RF puncture signal is above the threshold at 715, present control output is saved as a feedforward setting for power control mode at 718, and the controller is changed to power control mode at 720. In one embodiment, the setpoint is set to a power-based threshold value, such as Pₗᵢₘᵢₜ at 722. The control performs a control action on the RF puncture signal at 716, and therapy continues in power control mode.

A determination is made as to whether the delivery of the RF puncture signal is complete at 724. If delivery continues, the puncture signal is monitored, and feedback control of the puncture signal is performed at a return to 706.

If the controller 212 is in power control mode at 726, a determination is made as to whether the impedance in the patient circuit is above a threshold amount at 728, such as whether Zₚₐₜᵢₑₙₜ>(Vₐᵣ)²/Pₗᵢₘᵢₜ. For example, the measurement circuit 210 provides voltage and current measurements to the controller 212, which determines the impedance in the patient circuit from the voltage and current measurements and compares the measured impedance to the square of the measured voltage divided by a power threshold value or Pₗᵢₘᵢₜ in the embodiment (after the debounce or settling period). If the measured impedance is below the threshold at 730, the control performs a control action on the RF puncture signal at 716, and therapy continues in power control mode.

If the impedance is above the threshold at 732, present control output is saved as a feedforward setting for voltage control mode at 734, and the controller is changed to voltage control mode at 736. In one embodiment, the setpoint is set to a voltage-based threshold value, such as Vₛₑₜₚₒᵢₙₜ, such as a setpoint for a desired steady state voltage amount at 738. The control performs a control action on the RF puncture signal at 716, and therapy continues in voltage control mode.

Again, determination is made as to whether the delivery of the RF puncture signal is complete at 724. If delivery continues, the puncture signal is monitored, and feedback control of the puncture signal is performed at a return to 706. Otherwise, the third method 700 is ended at 740.

Additionally, the method 300 of FIG. 3 monitors parameters of the RF puncture delivery to avoid the tissue desiccation and tissue fulguration regions at 306, such as during a stage limiting power at 304 and after creating a puncture. In some embodiments of electrosurgical devices, real electrical power and apparent electrical power in an RF puncture signal are generally equal during tissue puncture, which lasts a limited amount of time. After puncture, real power and apparent power diverge such as apparent power is distinct from and greater than real power in the RF puncture signal. In one example, real power and apparent power are relatively high during a puncture of tissue. After the puncture of tissue has been formed, and the puncture electrode is in the blood pool (encapsulated by an electrically insulative bubble of gas, in some examples), the real power becomes relatively lower than the apparent power and lower than the real power before the puncture. In example of the electrosurgical system 100 in an abnormal state during delivery of the RF puncture signal, the real power does not drop during after the cut.

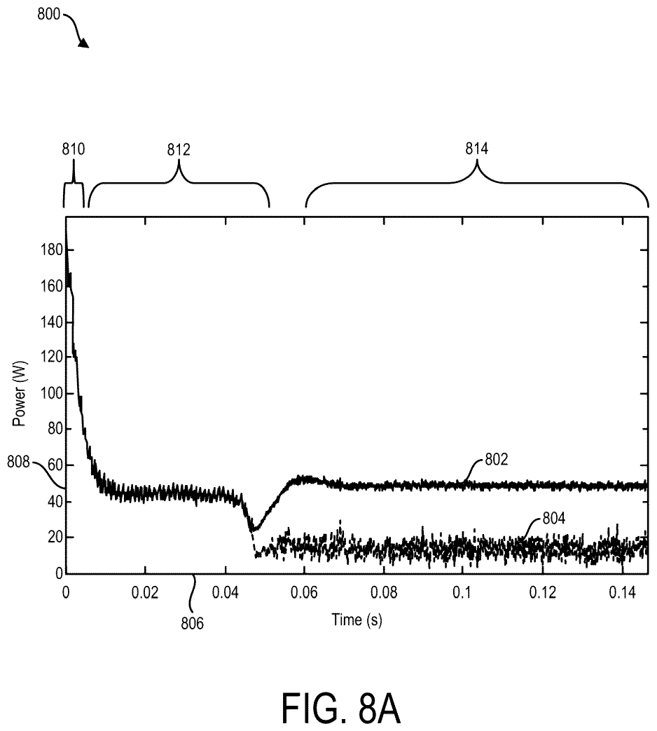

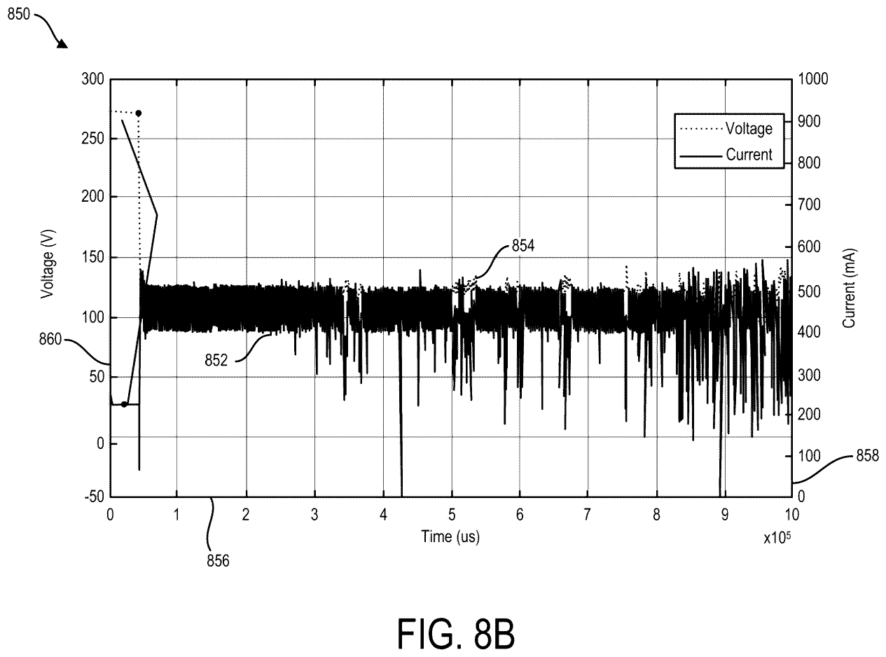

FIG. 8A illustrates a graph 800 including a plot of apparent electrical power 802 and real electrical power 804 of an RF puncture signal with respect to time as measured with the electrosurgical generator 200 during a tissue puncture procedure with a transseptal guidewire 106. A timeline, or time since activation of the puncture signal in the puncture procedure in seconds is 806 is presented along an x-axis. Electrical power is presented along a y-axis 808. For the purposes of illustration, the puncture signal delivery is generally separated into three stages with respect to time, a ramp up stage 810 during the application of the feedforward setting, a puncture stage 812 during tissue puncture, and a final steady state stage 814 subsequent the puncture. During the puncture stage 812, which lasts about 200-300 milliseconds (ms) into the puncture procedure, real power 802 and apparent power 804 are approximately the same in a normal delivery of the RF puncture signal. After the puncture stage 812, the apparent power 802 and real power 804 plots diverge in which real power 804 is less than apparent power 802 in the steady stage 814.

FIG. 8B illustrates a graph 850 including a plot of electrical current magnitude 852 and voltage magnitude 854 of a puncture signal with respect to time as measured by a measurement circuit 210 of an electrosurgical generator 200 during a tissue puncture procedure with a transseptal guidewire 106. A timeline, or time since activation of the puncture signal in the puncture procedure in seconds is 856 is presented along an x-axis. Current in amperes (A) root-mean-square (RMS) is presented along a right-side y-axis 858, and voltage in volts (V) RMS is presented along the left-side y-axis 860. In the illustrated example of the RF puncture procedure, the power limiting at 304 of method 300 have been disabled and the RF puncture signal is an abnormal state with an overheated tissue-electrode interface that results in tissue fulguration. The graph 850 illustrates high current delivery 852 with relatively low voltage delivery 854 shortly after the start of the RF puncture signal delivery.

In one example, a cut is formed in the tissue within about 200 milliseconds to 300 milliseconds from the beginning of the delivery of the RF puncture signal with the feedforward control setting at 302 of method 300. If the real power and apparent power have not diverged after such time, the RF puncture signal could produce thermal energy at the electrode-tissue interface in the tissue fulguration temperature region. Similarly, if the RF puncture signal includes a relatively high current and low voltage, the RF puncture signal could produce thermal energy at the electrode-tissue interface in the tissue fulguration temperature region. In one embodiment, the controller 212 is configured to monitor time, voltage, and current at 306, and terminate the RF puncture signal if the parameters indicate an abnormal state. Indications of an abnormal state include a lack of divergence of the real power and apparent power after a threshold period of time, lack of change in real power after a threshold period of time, high current after a threshold period of time. The controller 212 in some embodiments is also configured to measure temperature with the thermal sensor 142, and to terminate the RF puncture signal if temperature indicates the likelihood of tissue fulguration.

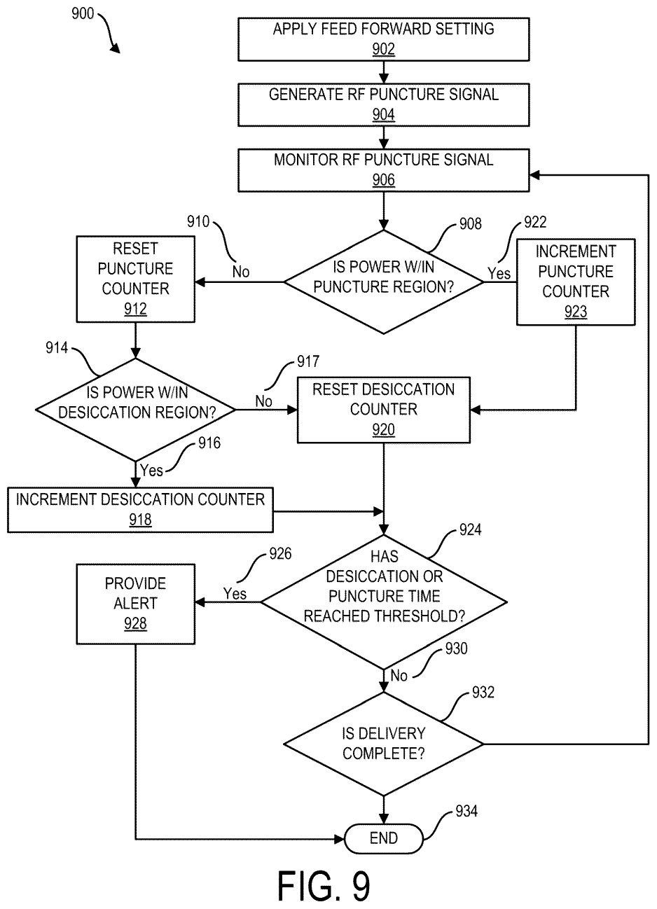

FIG. 9 illustrates a method 900, which is an embodiment of method 300, that includes an embodiment of a scheme to determine if the RF puncture delivery is in abnormal state, such as at 306. In the embodiment, the RF puncture signal is terminated if the duration of the cut appears to have exceeded a cut target time threshold. In such as circumstance, the method 900 avoids tissue fulguration. Also, the RF puncture signal is terminated if power, such as real electrical power, is in a tissue desiccation region for longer than a desiccation target time threshold. In such a circumstance, the method 900 avoids tissue desiccation.

A control setting matching a desired steady state delivery of the RF puncture signal is selected at 902. The steady state delivery is a steady state voltage, in one embodiment, the steady state voltage is applied as a voltage setpoint. The voltage setpoint corresponds with a feedforward control setting. The control setting is applied, which is now labeled the original control setting, and the RF puncture signal is generated at 904. The puncture signal is monitored, and feedback control of the puncture signal is performed at 906. As illustrated, method 900 generates an RF puncture signal at 302 and can be configured to dynamically control the RF output at 304 in method 300 with an electrosurgical generator 200.

In the illustrated embodiment, an amount of real power of the RF puncture signal is associated with an ability of the RF puncture signal to puncture tissue. For example, the real power with an ability to puncture tissue is set as a region of real power having a first real power value, or puncture ceiling threshold, and a second real power value, or real power floor threshold. Real power values between the real power floor and real power ceiling thresholds are in a real power puncture region. Additionally, an amount of real power is associated with a likelihood of desiccating tissue. For example, the real power likely to desiccate tissue is an amount of real power below a real power value designated as a real power desiccation threshold. Real power values lower than the real power desiccation threshold are in a real power desiccation region. In some embodiments, the real power floor threshold is the same as the real power desiccation threshold. Further, an amount of time is associated with a desiccation time, which is an amount of continuous time of real power in the real power desiccation region likely to produce tissue desiccation. Also, an amount of time is associated with a puncture time, which is an amount of continuous time of real power in the real power puncture region. If a puncture signal has spent more time than the puncture time, the system is likely in an abnormal state.

A determination is made as to whether the amount of real power of the RF puncture signal is within the real power region at 908. If the real power of the RF puncture signal is outside of the real power region at 910, a cut counter is reset at 912 and a determination is made as to whether the real power of the RF puncture signal is within the real power desiccation region at 914. If yes at 916, a desiccation counter is incremented at 918. If no at 917, the desiccation counter is reset at 920. If the real power of the RF puncture signal is within the real power region at 922, a puncture duration counter is incremented at 923 and the desiccation counter is reset at 920.

A determination is made as to whether the desiccation time or puncture time have been exceeded at 924. For example, the desiccation time and puncture times can be determined via accessing the desiccation counter and puncture counter, which counters can be related to the passing of time. If the desiccation time or puncture time have been exceeded at 926, the RF puncture signal is terminated, and an alarm is issued at 928. If the desiccation time and puncture time have not been exceeded at 930, a determination is made as to whether delivery continues at 932. If so, the puncture signal is monitored, and feedback control of the puncture signal is performed at 906. Otherwise, the method 900 is ended at 934.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A radiofrequency (RF) generator for use in a tissue puncture system for puncturing a tissue in a body, the RF generator comprising:
an RF energy source having an active connector and a return connector, the active connector configured to be coupled to an active electrode of an RF puncture device and the return connector configured to be coupled to a return electrode, the active electrode and return electrode configured to couple to the body; and
a controller coupled to the RF energy source, the controller configured to:
apply an RF puncture signal based on a feedforward control setting,
assert an electrical power control of the puncture signal based on thermal energy to the tissue applied with the RF puncture signal, and
monitor the puncture signal for desiccation and fulguration of the tissue based on thermal energy applied to the tissue with the RF puncture signal.

2. The RF generator of claim 1, wherein the feedforward control setting is based on a nominal feedforward control setting stored in a non-transitory memory device.

3. The RF generator of claim 2, wherein the feedforward control setting is an adjusted nominal feedforward control setting.

4. The RF generator of claim 1, comprising an RF output circuit coupled to the controller and wherein the electrical power control is asserted via disabling an RF output circuit.

5. The RF generator of claim 4, wherein the RF output circuit includes a voltage converter, and the voltage converter is disabled based on electrical current drawn by the voltage converter.

6. The RF generator of claim 5, wherein the RF output circuit includes a comparator circuit coupled to the voltage converter, and the electrical current and a reference current value are provided to a comparator circuit.

7. The RF generator of claim 5, comprising a measurement circuit coupled to the RF output circuit and to the controller, wherein the measurement circuit is applied to determine the electrical current and provide a digitized signal to the controller.

8. The RF generator of claim 4, wherein the electrical power is one of an apparent electrical power or a real electrical power.

9. The RF generator of claim 4, wherein the electrical power control is asserted via disabling the RF output circuit based on a thermal signal.

10. The RF generator of claim 9, comprising a thermal sensor connector and wherein the RF puncture device includes a thermal sensor proximate the active electrode.

11. The RF generator of claim 10, comprising a measurement circuit coupled to the thermal sensor connector and to the controller.

12. The RF generator of claim 1, wherein the controller is configured to assert the electrical power based on one of a power of the RF puncture signal or a voltage of the RF puncture signal.

13. The RF generator of claim 1, wherein the controller is configured to monitor the puncture signal for desiccation based on time of insufficient power of the RF puncture signal.

14. The RF generator of claim 1, wherein the controller is configured to monitor the puncture signal for fulguration based on time of sufficient power of the RF puncture signal.

15. The RF generator of claim 1, wherein the RF puncture device is an electrosurgical transseptal guidewire.

16. A radiofrequency (RF) generator for use in a tissue puncture system for puncturing a tissue in a body, the RF generator comprising:
an RF energy source having an active connector and a return connector, the active connector configured to be coupled to an active electrode of an RF puncture device and the return connector configured to be coupled to a return electrode, the active electrode and return electrode configured to couple to the body; and
a controller coupled to the RF energy source, the controller configured to:
apply an RF puncture signal based on a feedforward control setting,
assert an electrical power control of the puncture signal based on thermal energy to the tissue applied with the RF puncture signal from one of a power of the RF puncture signal or a voltage of the RF puncture signal, and
monitor the puncture signal for desiccation and fulguration of the tissue based on thermal energy applied to the tissue with the RF puncture signal, wherein the controller is configured to monitor the puncture signal for desiccation based on time of insufficient power of the RF puncture signal, and wherein the controller is configured to monitor the puncture signal for fulguration based on time of sufficient power of the RF puncture signal.

17. The RF generator of claim 16, comprising an RF output circuit coupled to the controller and wherein the electrical power control is asserted via disabling an RF output circuit.

18. The RF generator of claim 16, wherein the feedforward control setting is based on a nominal feedforward control setting stored in a non-transitory memory device.

19. A method of use in a radiofrequency (RF) generator in a tissue puncture system, the RF generator including an RF energy source having an active connector and a return connector, the active connector configured to be coupled to an active electrode of an RF puncture device and the return connector configured to be coupled to a return electrode, the active electrode and return electrode configured to couple to a body, the method comprising:
applying an RF puncture signal based on a feedforward control setting;
asserting an electrical power control of the puncture signal based on thermal energy to the tissue applied with the RF puncture signal; and
monitoring the puncture signal for desiccation and fulguration of the tissue based on thermal energy applied to the tissue with the RF puncture signal.

20. The method of claim 19, wherein the puncture signal is monitored for desiccation based on time of insufficient power of the RF puncture signal for fulguration based on time of sufficient power of the RF puncture signal.
